(19) United States
(12) Patent Application Publication
(10) Pub. No.: US 2021/0100545 A1
(43) Pub. Date: Apr. 8, 2021

(54) SUTURE ANCHOR

(71) Applicant: Valeris Medical, Inc., Marietta, GA (US)

(72) Inventor: Brendan D. Thies, Woodstock, GA (US)

(21) Appl. No.: 16/594,761

(22) Filed: Oct. 7, 2019

(51) Int. Cl. A61B 17/04; A61F 2/08

(52) U.S. Cl. CPC A61B 17/0401; A61F 2/0811; A61B 2017/0409; A61F 2002/30761; A61F 2002/0841; A61F 2002/0852; A61F 2002/0888; A61B 2017/0414

(57) ABSTRACT

A suture anchor has a cylindrical body and an integral entry tip. The entry tip is at a distal end and the cylindrical body has a hollow proximal end portion and projections formed on an external surface of the body for retaining the anchor in a pre-drilled hole formed in bone. Two suture openings are formed extending through the body to opposed sides of the cylindrical body between the proximal end of the body and the entry tip at the distal end. Each opening is connected by slots or recesses of the body. A suture entering the hollow proximal end portion passing out through a first opening extends externally along a slot and enters a second opening extending out the second opening on an opposite side extending along an opposing slot and reentering the opposite side first opening and extending back out the hollow proximal end portion.

### Field of the Invention

The present invention relates to a suture anchor device for attaching soft tissue to bone.

### BACKGROUND OF THE INVENTION

When soft tissue such as a ligament or a tendon becomes detached from a bone, surgery may be indicated to reattach or reconstruct the tissue. Various fixation devices, including sutures, screws, staples, wedges and plugs have been used in the past to secure soft tissue to bone.

More recently, various types of suture anchors have been developed. The suture anchors are implanted in bone, and suture passed through the soft tissue is secured to the suture anchor. The technique usually requires the surgeon to tie knots in the suture, though various knotless techniques are also available.

Older style bone anchors had an external eyelet to allow the sutures to be fed through after the screw was driven into the bone. This left the eyelet exposed in the soft muscle tissue at a proximal end of the device.

Later versions of bone anchors have the sutures inserted inside the screw tip well inside the bone. This allows the outer end of the screw to stay flush with the cortical bone outer surface. The sutures can pass inside in some hollow screws from near the tip outer surface. Others have the sutures held near the tip and are exterior held between the anchor threads and the bone. In some suture anchor systems, there is a self-driving feature that has a stainless steel punch with a steel tip that can be malleted into the bone. This design leaves the tip end trapped in the bone opening meaning the patient has a small metal tip in his shoulder or knee.

Ideally, the anchor should leave no metal remnants and ideally is able to provide a knotless fixation or hold of the suture on insertion into the bone.

### SUMMARY OF THE INVENTION

A suture anchor has a cylindrical body with projections and a pair of suture openings. The cylindrical body has an entry tip at a distal end portion and a hollow tool attachment portion at a proximal end portion. The projections are formed on the external surface of the cylindrical body for retaining the anchor in a predrilled hole formed in bone. The pair of suture openings extend through the cylindrical body. A first opening is in the hollow tool attachment portion. The second opening is below the hollow tool attachment portion. Both first and second openings are circumferentially aligned and spaced longitudinally relative to a length of the cylindrical body. The anchor allows a suture to be passed through the first opening from the hollow tool attachment portion externally and though the second opening and back into the first opening on an opposite side of the cylindrical body back into the hollow tool attachment portion.

The first opening and second opening are parallel and pass crossing a longitudinal axis of the cylindrical body through the exterior surface of the cylindrical body. The first and second openings are configured to allow the suture to slide freely when held in the cylindrical body.

The cylindrical body further may have a pair of slots, a first slot extending from the first opening to the second opening on one side of the cylindrical body, a second slot extending from the first opening to the second opening on an opposite side. Each slot is configured to form a recess along the longitudinal space between the first opening and second opening into which the suture can be positioned external of the cylindrical body.

In a second embodiment, the distal end portion has an opening for receiving one or more additional sutures. The opening is one of round square or oval.

The hollow tool attachment portion has an opening configured for receiving a shaft of an anchor driving device.

A suture anchor assembly has a driver, a length of suture and a suture anchor. The driver has a hollow driver shaft for receiving the suture. The hollow driver shaft has a distal end configured to hold the suture anchor. The suture anchor has a cylindrical body. The cylindrical body has an entry tip at a distal end portion and a hollow tool attachment portion at a proximal end portion. The projections are formed on the external surface of the cylindrical body for retaining the anchor in a pre-drilled hole formed in bone. The pair of suture openings extend through the cylindrical body. A first opening is in the hollow tool attachment portion. The second opening is below the hollow tool attachment portion. Both first and second openings are circumferentially aligned and spaced longitudinally relative to a length of the cylindrical body. The anchor allows a suture to be passed through the first opening from the hollow tool attachment portion externally and though the second opening and back into the first opening on an opposite side of the cylindrical body back into the hollow tool attachment portion.

The length of suture has a first end and a second end wherein the suture is held in the suture anchor free to slide and extend through the hollow tool attachment portion inside the hollow driver shaft.

In the second embodiment, the suture anchor distal end portion has an opening for receiving one or more additional sutures. The second embodiment suture anchor further has a second length of suture passing through the opening in the distal end portion.

A method of implanting a suture anchor has the steps of: threading a length of suture through a proximal opening out through a first opening along an exterior slot into a second opening and out and through an opposing side along an outside slot back into the first opening and into the proximal end opening; adjusting the length of suture until the ends of the suture substantially aligned; passing the ends of the length of suture into a hollow shafted driver device; and attaching the anchor with threaded suture to the driver for implantation. The method further has the steps of: implanting the threaded suture anchor into a pre-drilled bone; detaching the driver leaving the ends of the length of suture exposed above the bone; and wherein the length of suture is slidable relative to the suture anchor for adjustment. The method can further have the steps of: attaching a ligament, cartilage or other soft tissue to the length of suture and knotting the suture about the anchor. The method wherein the suture anchor has a suture holding opening at a distal end and the method further comprises threading a second length of suture through the opening at the distal end and along the exterior of the suture anchor.

### BRIEF DESCRIPTION OF THE DRAWINGS

The invention will be described by way of example and with reference to the accompanying drawings in which:

FIG. 1 is a side plan view of a first embodiment anchor of the present invention.

FIG. 2 is a perspective view of the first embodiment.

FIG. 3 is a second perspective view of the first embodiment anchor showing the proximal end.

FIG. 4 is an opposite end view showing the internal surface of the tip or distal end and the central opening of the hollow cylindrical body of the first embodiment anchor.

FIG. 5 is a cross-sectional view of the first embodiment anchor.

FIG. 6 is a side view of the first embodiment anchor with a suture going through the cylindrical body.

FIG. 7 is a schematic view showing the first embodiment anchor with a suture passed through the soft tissue driven into bone.

FIG. 8 is a side view of the first embodiment anchor attached to a driver shaft.

FIG. 9 is a schematic view showing the first embodiment anchor attached to a driver and passed through the soft tissue driven into bone.

FIG. 10 is a second view of the first embodiment anchor taken from FIG. 9.

FIG. 11 is a side view of the first embodiment anchor with the driver being removed showing the suture extending from the driver shaft.

FIG. 12 is a side plan view of a second embodiment anchor of the present invention with a distal opening.

FIG. 13 is a perspective proximal view of the second embodiment anchor.

FIG. 14 is a cross-sectional view of the second embodiment anchor.

FIG. 15 is a second cross-sectional view of the second embodiment anchor.

FIG. 16 is a perspective distal view of the second embodiment anchor.

FIG. 17 is a side view of the second embodiment anchor with a suture going through the cylindrical body.

FIG. 18 is an opposite side view taken from FIG. 17 of the second embodiment anchor with a suture going through the cylindrical body.

FIG. 19 is a side view of the second embodiment anchor attached to a driver shaft.

FIG. 20 is a schematic view showing the second embodiment anchor attached to a driver and passed through the soft tissue driven into bone.

FIG. 21 is a side view of the second embodiment anchor with a first length of suture going through the cylindrical body and a second length of suture going through the opening in the distal end portion.

FIG. 22 is an opposite side view taken from FIG. 21 of the second embodiment anchor with a first length of suture going through the cylindrical body and a second length of suture going through the opening in the distal end portion.

FIG. 23 is a schematic view showing the second embodiment anchor with two lengths of suture attached to the anchor and passed through the soft tissue driven into bone.

FIG. 24 is a schematic view showing the second embodiment anchor attached to a driver.

FIG. 25 is a side plan view of the driver handle taken from FIG. 24.

FIG. 26 is a schematic view showing the second embodiment anchor attached to the driver shaft with a length of suture passing through the opening in the distal end portion and along the driver shaft, the anchor being passed through soft tissue into bone.

FIG. 27 is a schematic view taken from FIG. 26 showing the driver shaft being removed leaving the length of suture passed through the opening in the distal end portion and a length of suture passed through the cylindrical body and inside the driver shaft.

### DETAILED DESCRIPTION OF THE INVENTION

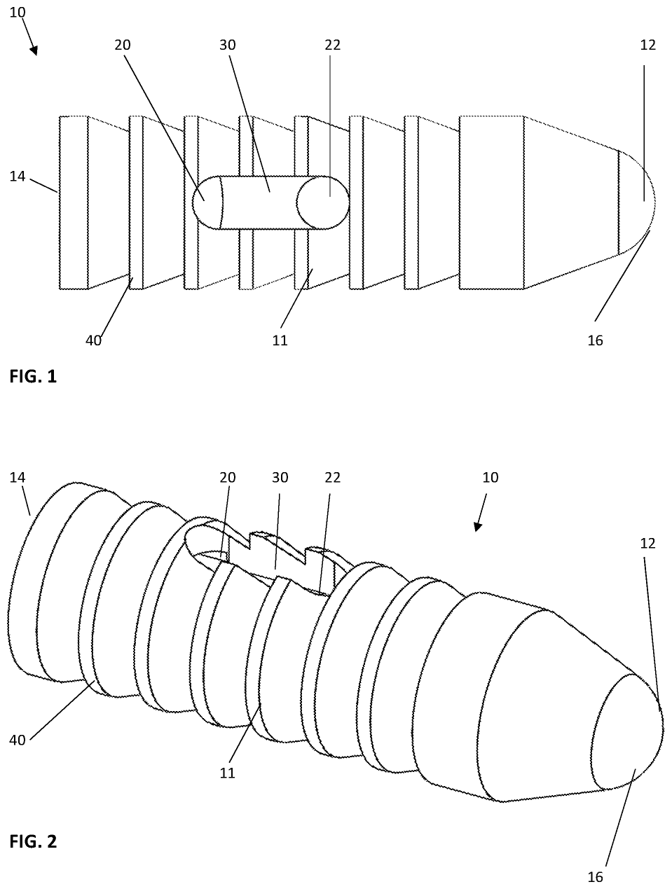

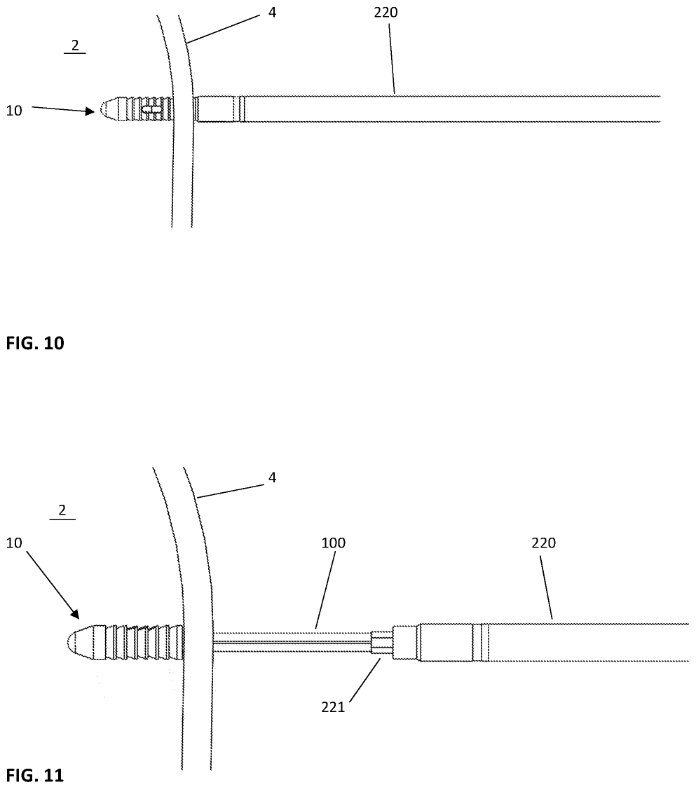

With reference to the drawings, FIGS. 1-11 depict a first embodiment suture anchor 10 of the present invention. As shown, the suture anchor 10 has a distal end 12 and a proximal end 14. The suture anchor 10 has a cylindrical body 11 with a plurality of projections 40 for retaining the suture anchor 10 in bone. Shown in FIG. 1 are a pair of openings, a first opening 20 extends through the suture anchor 10 in the hollow tool attachment portion 50. Extending longitudinally along the suture anchor 10 are two opposing slots 30 which extend distally toward a second opening 22. The second opening 22 is longitudinally aligned and parallel to the first opening 20 and is spaced longitudinally by a short distance.

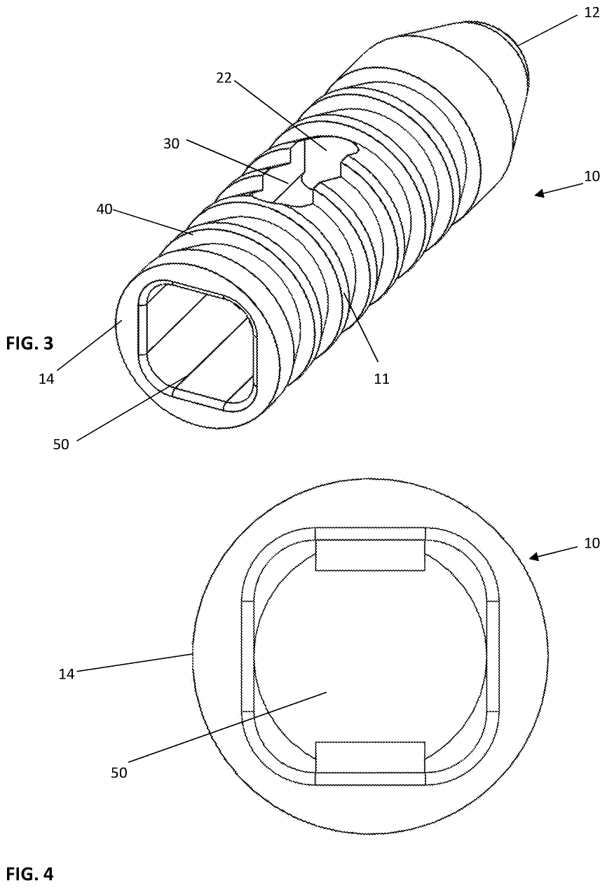

FIG. 2 shows a perspective view with an entry tip 16 at the distal end 12 of the suture anchor 10. FIG. 3 shows a perspective view of the suture anchor 10 showing the hollow tool attachment opening 50 at the proximal end 14. FIG. 4 is an enlarged view of the hollow tool attachment opening 50 at the proximal end 14.

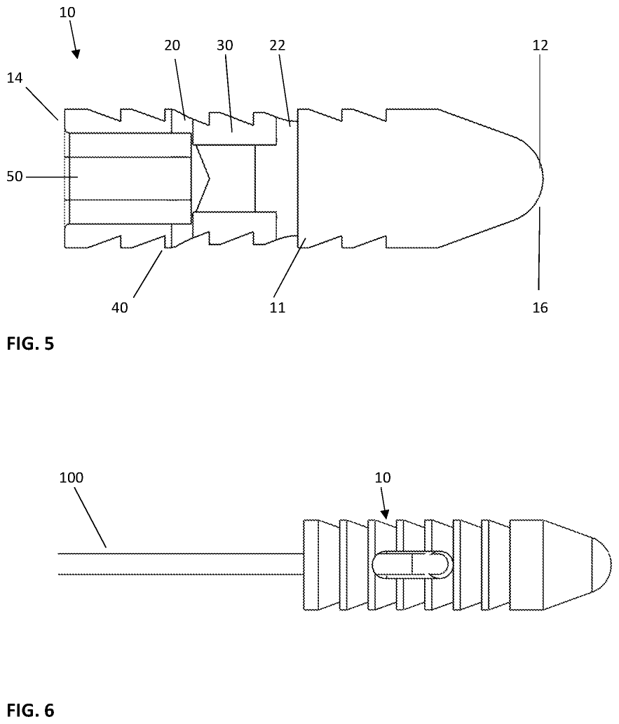

FIG. 5 is a cross-sectional view of the suture anchor 10. In this view, the first opening 20 is shown intersecting the hollow tool attachment opening 50 and the slot 30 extending between the two openings 20, 22.

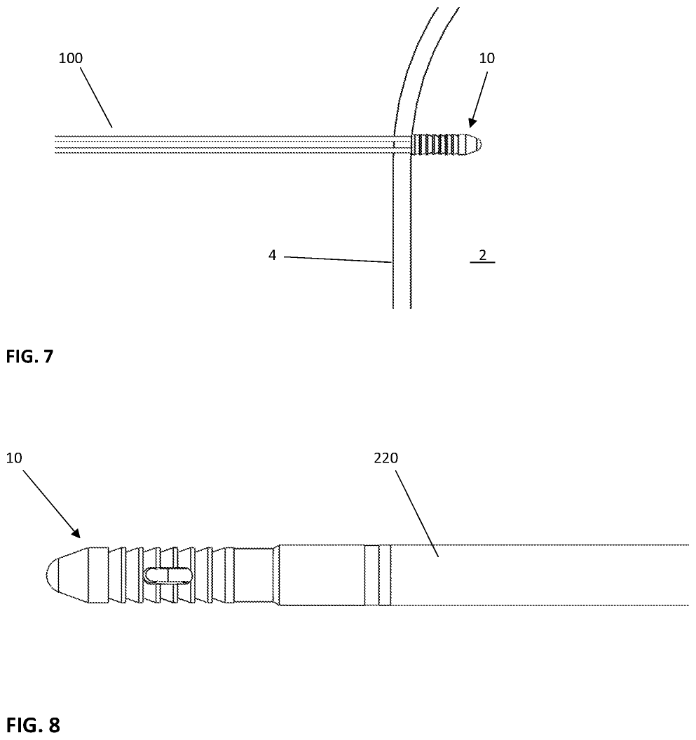

FIG. 6 illustrates a suture 100 that has been positioned inside the proximal end 14 of the suture anchor 10. In positioning the suture 100 into the suture anchor 10, an end of the suture 100 is inserted through the opening 50 extending outwardly through the first opening 20 within the slot 30 and is then fed back through the second opening 22 extending therethrough to the opposite side opposing slot 30 back into the first opening 20 thereby looping around the entire suture anchor 10. When this occurs the suture 100 can slide freely through the suture anchor 10 such that the ends of the suture 100 can be approximately equal if so desired, this is best illustrated in FIG. 7 wherein the push in suture anchor 10 has been pushed through soft tissue 4 into bone 2.

During assembly, this pushing of the suture anchor 10 into the bone 2 occurs after a pre-drilled hole is made in the bone 2. As illustrated in FIGS. 8-11, the suture anchor 10 is positioned with a driver shaft 220 having an end 221.

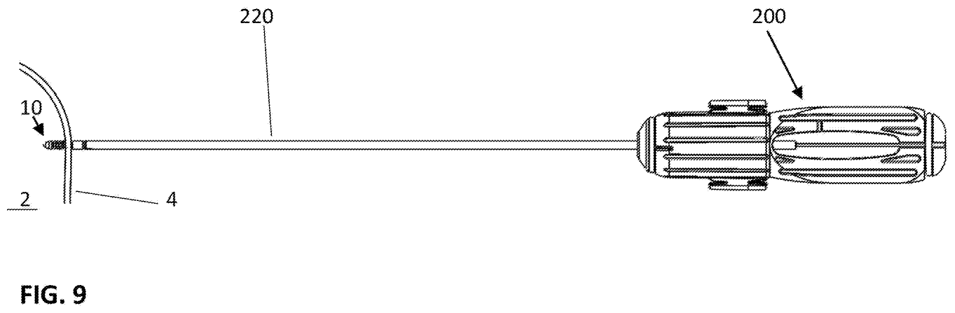

The driver device 200 with driver shaft 220 and suture anchor 10 attached is shown in FIG. 9. The suture 100 is positioned inside the hollow driver shaft 220 and extends outwardly through the driver device 200 handle.

As shown in FIG. 11, once the suture anchor 10 is positioned in the bone 2, the suture anchor 10 can be detached from the driver 200 simply by pulling back the driver device 200. This is best illustrated in FIG. 25, wherein the driver shaft 220 with suture 100 is shown on the left-hand side 202 of the driver device 200 and the suture 100 is shown entering internally of the handle 204 and out the proximal end of the handle 204 and then wrapped about attachment features 205, 206. Upon release of the suture 100, the suture 100 can simply be unwound from the attachment features 205, 206 and allow the driver device 200 to slip away from the anchor 10 as shown in FIG. 11. When this occurs, the suture 100 will be available to attach soft tissue if so desired or provide an opportunity for the surgeon to provide a slip knot to hold any ligament or cartilage or the soft tissue, if so desired.

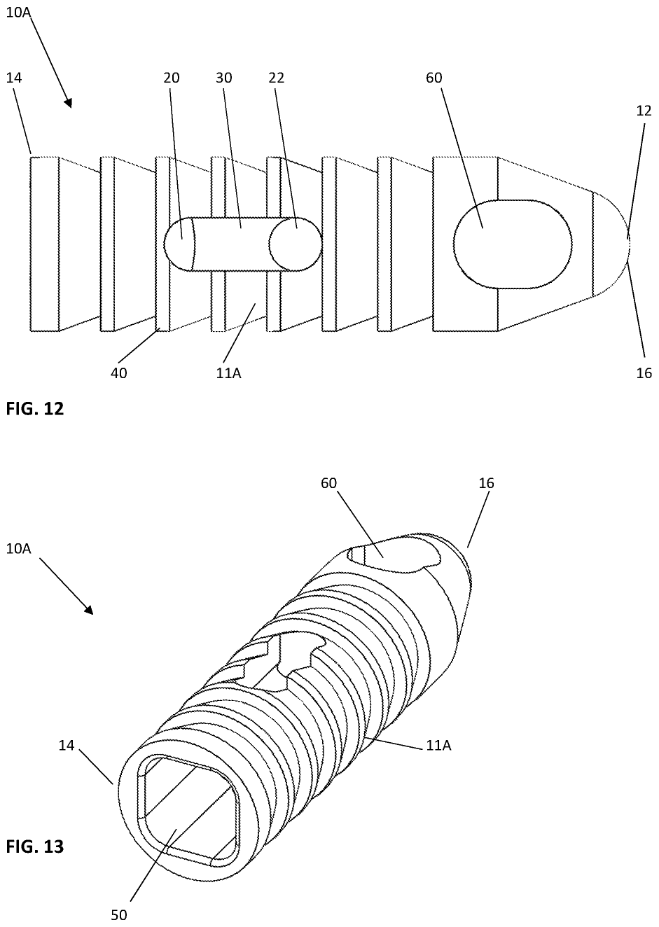

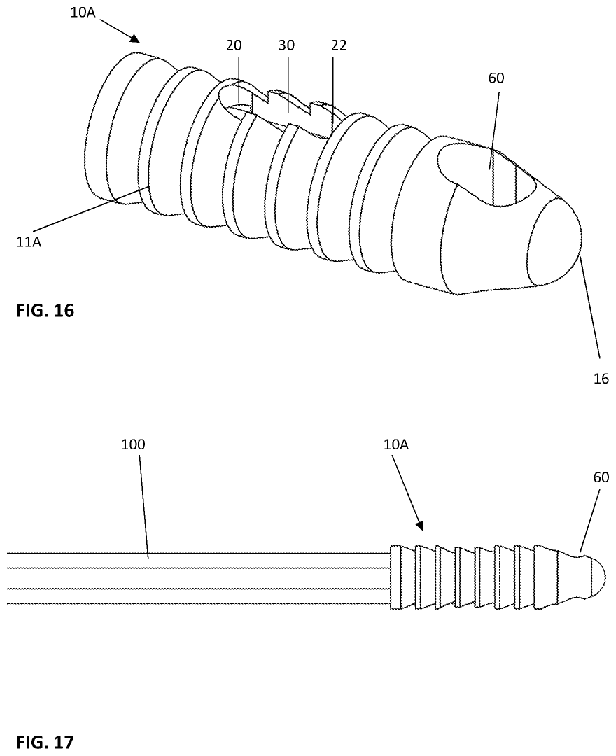

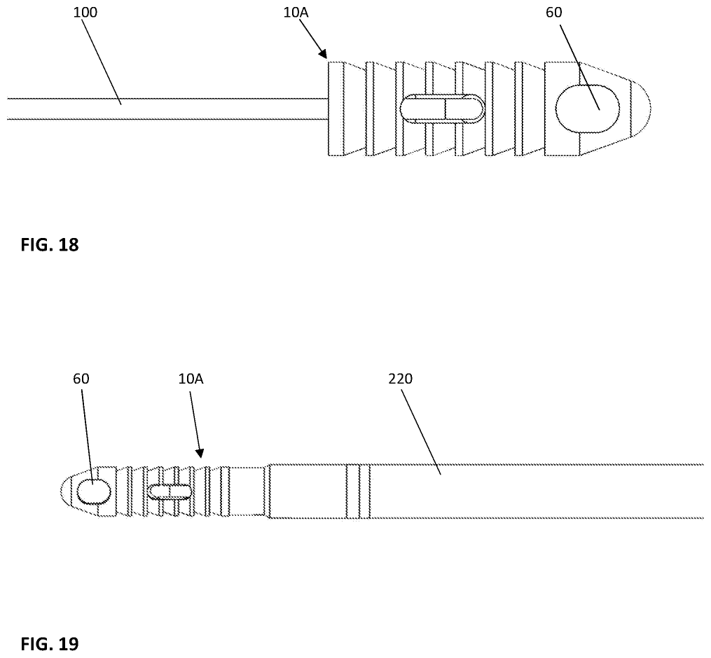

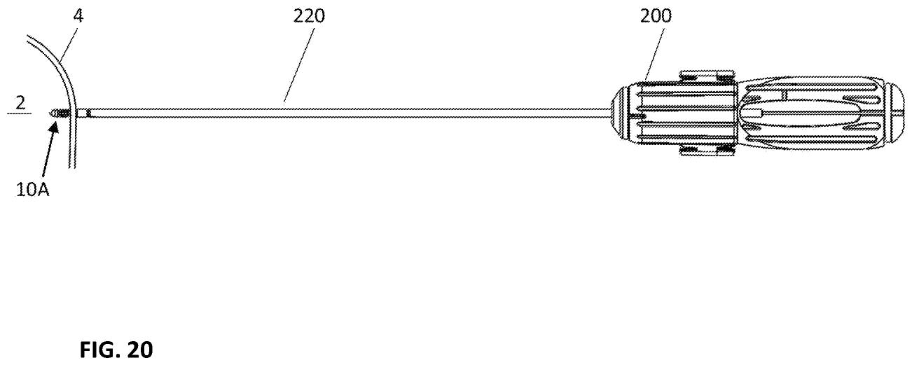

With reference to FIGS. 12-23, a second embodiment suture anchor 10A is illustrated. The suture anchor 10A is identical to the suture anchor 10 with the exception that at the distal end 12 is an enlarged opening 60 near the entry tip 16. This enlarged opening 60 provides a way in which one or more sutures can be passed through the opening 60.

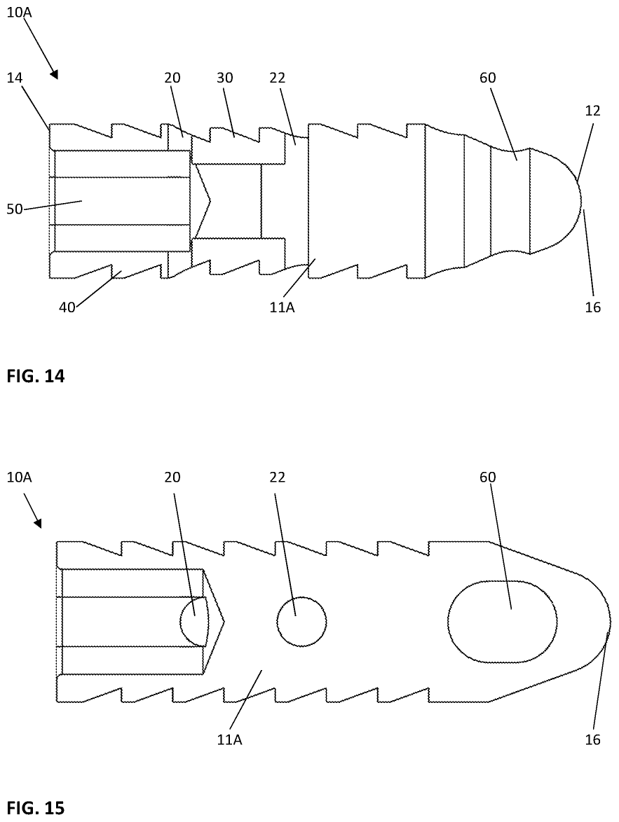

As shown in FIGS. 14 and 15 in cross-sectional views, the enlarged opening 60 is somewhat oval shaped as shown most clearly in FIG. 15. As described before, the first opening 20 and second opening 22 extend through the suture anchor cylindrical body 11, with the first opening 20 passing through a portion of the hollow tool attachment opening 50 identical to the first embodiment suture anchor 10.

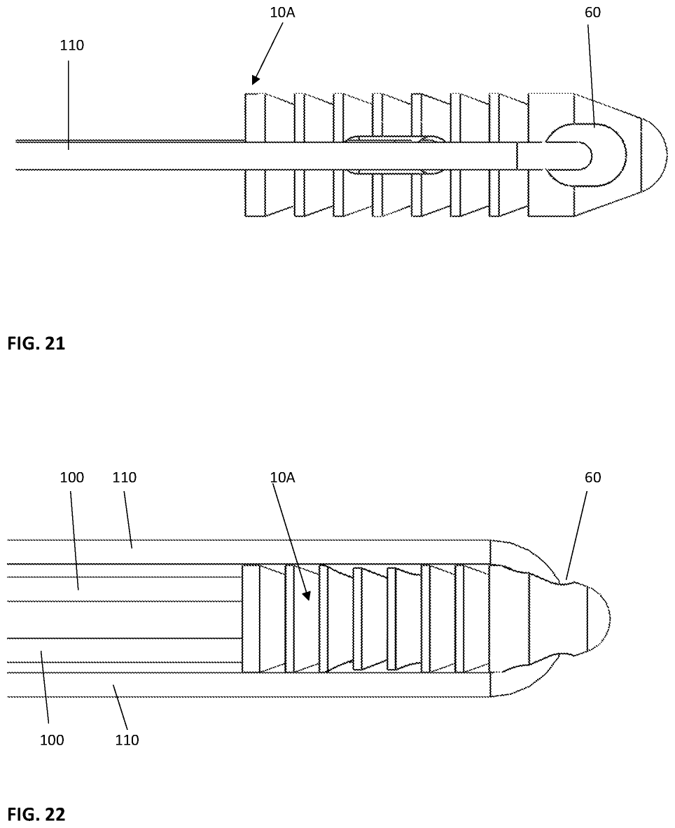

As shown in FIG. 17 the suture 100 can be passed through the suture anchor 10A in a similar fashion as was earlier explained in the first embodiment suture anchor 10. All of the remaining views 18-21 are substantially identical to the views of the first embodiment, with the exception that external the cylindrical body 11A of suture anchor 10A at least one suture is passed through the enlarged opening 60 and extends externally along the sides of the suture anchor 10A. This is best illustrated in FIGS. 21-23.

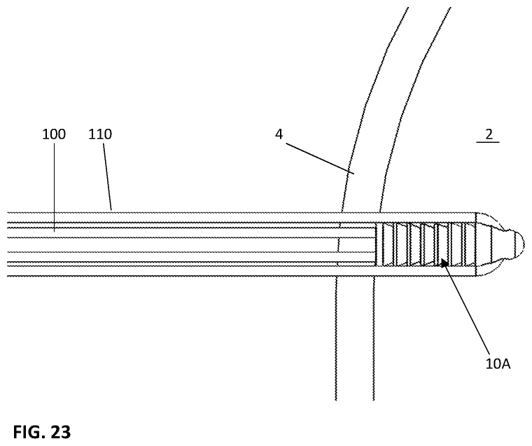

As shown, the second suture 110 can be passed through the opening 60 along each side of the suture anchor 10A and compressed between the suture anchor 10A and the bone 2, as illustrated in FIG. 23. This ability to secure more than one suture using the alternative embodiment suture anchor 10A provides the surgeon with more opportunities to tie the soft tissue, ligaments, or cartilage to the bone when the suture anchor is inserted.

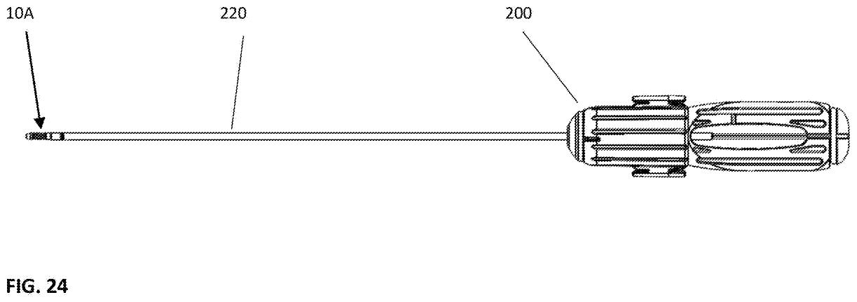

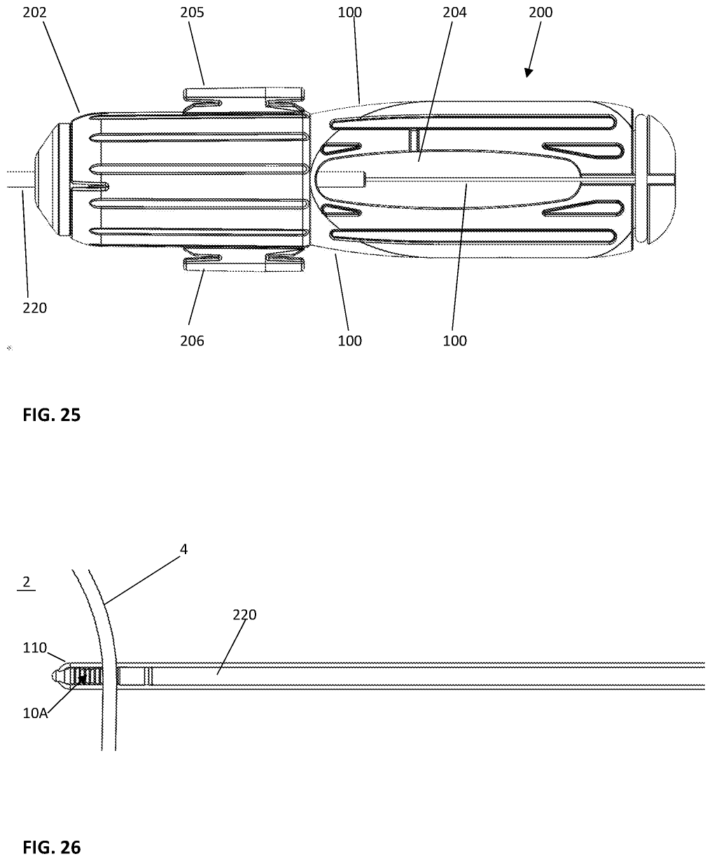

FIG. 24 shows the suture anchor 10A mounted on the driver device 200 having a hollow driver shaft 220 as previously described.

FIG. 26 shows the suture anchor 10A with suture 110 inserted through soft tissue 4 and into bone 4 into a pre-drilled hole.

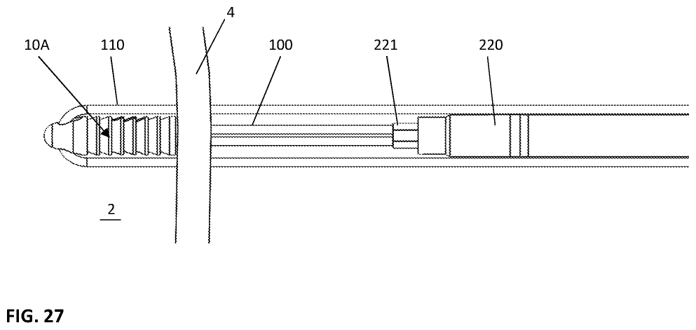

In FIG. 27, the driver shaft 220 with tip 221 is shown detached from the suture anchor 10A that is inserted through soft tissue 4 into bone 2 with sutures 110 extending externally and sutures 100 extending internal of the driver shaft 220.

A method of implanting a suture anchor has the steps of: threading a length of suture through a proximal opening out through a first opening along an exterior slot into a second opening and out and through an opposing side along an outside slot back into the first opening and into the proximal end opening; adjusting the length of suture until the ends of the suture substantially aligned; passing the ends of the length of suture into a hollow shafted driver device; and attaching the anchor with threaded suture to the driver for implantation. The method further has the steps of: implanting the threaded suture anchor into a pre-drilled bone; detaching the driver leaving the ends of the length of suture exposed above the bone; and wherein the length of suture is slidable relative to the suture anchor for adjustment. The method can further have the steps of: attaching a ligament, cartilage or other soft tissue to the length of suture and knotting the suture about the anchor. The method wherein the suture anchor has a suture holding opening at a distal end and the method further comprises threading a second length of suture through the opening at the distal end and along the exterior of the suture anchor.

Variations in the present invention are possible in light of the description of it provided herein. While certain representative embodiments and details have been shown for the purpose of illustrating the subject invention, it will be apparent to those skilled in this art that various changes and modifications can be made therein without departing from the scope of the subject invention. It is, therefore, to be understood that changes can be made in the particular embodiments described which will be within the full intended scope of the invention as defined by the following appended claims.

What is claimed is:

1. A suture anchor comprising:
a cylindrical body having an entry tip at a distal end portion and a hollow tool attachment portion at a proximal end portion;
projections formed on the external surface of the cylindrical body for retaining the anchor in a predrilled hole formed in bone;
a pair of suture openings extending through the cylindrical body, a first opening in the hollow tool attachment portion and a second opening below the hollow tool attachment portion, both first and second openings being circumferentially aligned and spaced longitudinally relative to a length of the cylindrical body; and
wherein a suture can be passed through the first opening from the hollow tool attachment portion externally and though the second opening and back into the first opening on an opposite side of the cylindrical body back into the hollow tool attachment portion.

2. The suture anchor of claim 1 wherein the cylindrical body further comprises:
a pair of slots, a first slot extending from the first opening to the second opening on one side of the cylindrical body, a second slot extending from the first opening to the second opening on an opposite side, each slot configured to form a recess along the longitudinal space between the first opening and second opening into which the suture can be positioned external of the cylindrical body.

3. The suture anchor of claim 1 wherein the distal end portion has an opening for receiving one or more additional sutures.

4. The suture anchor of claim 1 wherein the first opening and second opening are parallel and pass crossing a longitudinal axis of the cylindrical body through the exterior surface of the cylindrical body.

5. The suture anchor of claim 1 wherein the first and second openings are configured to allow the suture to slide freely when held in the cylindrical body.

6. The suture anchor of claim 1 wherein the hollow tool attachment portion has an opening for receiving a shaft of an anchor driving device.

7. The suture anchor of claim 3 wherein the opening in the distal end portion is one of round square or oval.

8. A suture anchor assembly comprises:
a driver having a hollow driver shaft for receiving a suture, the hollow driver shaft having a distal end configured to hold a suture anchor;
a suture anchor having a cylindrical body, the cylindrical body having an entry tip at a distal end portion and a hollow tool attachment portion at a proximal end portion; projections formed on the external surface of the cylindrical body for retaining the anchor in a predrilled hole formed in bone; a pair of suture openings extending through the cylindrical body, a first opening in the hollow tool attachment portion and a second opening below the hollow tool attachment portion, both first and second openings being circumferentially aligned and spaced longitudinally relative to a length of the cylindrical body; and wherein a suture can be passed through the first opening from the hollow tool attachment portion externally and though the second opening and back into the first opening on an opposite side of the cylindrical body back into the hollow tool attachment portion;
a length of suture having a first end and a second end wherein the suture is held in the suture anchor free to slide and extend through the hollow tool attachment portion inside the hollow driver shaft.

9. The suture anchor of claim 8 wherein the distal end portion has an opening for receiving one or more additional sutures.

10. The suture anchor of claim 9 further comprises a second length of suture passing through the opening in the distal end portion.

11. The suture anchor of claim 9 wherein the cylindrical body further comprises:
a pair of slots, a first slot extending from the first opening to the second opening on one side of the cylindrical body, a second slot extending from the first opening to the second opening on an opposite side, each slot configured to form a recess along the longitudinal space between the first opening and second opening into which the suture can be positioned external of the cylindrical body.

12. The suture anchor of claim 9 wherein the first opening and second opening are parallel and pass crossing a longitudinal axis of the cylindrical body through the exterior surface of the cylindrical body.

13. The suture anchor of claim 9 wherein the first and second openings are configured to allow the suture to slide freely when held in the cylindrical body.

14. The suture anchor of claim 9 wherein the hollow tool attachment portion has an opening for receiving a shaft of an anchor driving device.

15. The suture anchor of claim 9 wherein the opening in the distal end portion is one of round square or oval.

16. A method of implanting a suture anchor comprises the steps of:
threading a length of suture through a proximal opening out through a first opening along an exterior slot into a second opening and out and through an opposing side along an outside slot back into the first opening and into the proximal end opening;
adjusting the length of suture until the ends of the suture substantially aligned;
passing the ends of the length of suture into a hollow shafted driver device; and
attaching the anchor with threaded suture to the driver for implantation.

17. The method of claim 16 further comprises:
implanting the threaded suture anchor into a pre-drilled bone;
detaching the driver leaving the ends of the length of suture exposed above the bone; and
wherein the length of suture is slidable relative to the suture anchor for adjustment.

18. The method of claim 17 further comprises:
attaching a ligament, cartilage or other soft tissue to the length of suture and knotting the suture about the anchor.

19. The method of claim 16 wherein the suture anchor has a suture holding opening at a distal end and the method further comprises threading a second length of suture through the opening at the distal end and along the exterior of the suture anchor.
